(19) United States
(12) Patent Application Publication
(10) Pub. No.: US 2006/0211137 A1
(43) Pub. Date: Sep. 21, 2006

(54) Biosensor

(75) Inventors: Toshihide Ezoe, Kanagawa (JP); Toshiaki Kubo, Kanagawa (JP); Masashi Hakamata, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/362,354

(22) Filed: Feb. 27, 2006

(30) Foreign Application Priority Data
Feb. 28, 2005 (JP) 053449/2005

(51) Int. Cl. G01N 33/543; C12M 1/34

(52) U.S. Cl. 436/518; 435/287.2; 977/701

(57) ABSTRACT

It is an object of the present invention to provide a biosensor wherein a reference unit and a measurement unit can be prepared by performing only a single operation for immobilizing a physiologically active substance and wherein unnecessary electric charge does not remain in the reference unit. The present invention provides a biosensor which comprises a substrate composed of a metal surface or metal film coated with a hydrophilic polymer compound, and which has a surface for retaining a physiologically active substance and a surface that does not retain a physiologically active substance on a single plane of the substrate.

### TECHNICAL FIELD

The present invention relates to a biosensor and a method for analyzing an interaction between biomolecules using the biosensor. Particularly, the present invention relates to a biosensor which is used for a surface plasmon resonance biosensor and a method for analyzing an interaction between biomolecules using the biosensor.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The OCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

As a thin film having a functional group capable of immobilizing a physiologically active substance, there has been reported a measurement chip where a physiologically active substance is immobilized by using a functional group binding to metal, a linker with a chain length of 10 or more atoms, and a compound having a functional group capable of binding to the physiologically active substance (Japanese Patent No. 2815120). Moreover, a measurement chip comprising a metal film and a plasma-polymerized film formed on the metal film has been reported (Japanese Patent Laid-Open No. 9-264843).

When a specific binding reaction is measured between a physiologically active substance and a test substance, the test substance does not necessarily consist of a single component, but it is sometimes required to measure the test substance existing in a heterogeneous system, such as that in a cell extract. In such a case, if various contaminants such as proteins or lipids were non-specifically adsorbed on a detection surface, detection sensitivity in measurement would significantly be decreased. The aforementioned detection surface has been problematic in that such non-specific adsorption often takes place thereon. In order to solve such a problem, several methods have been studied. For example, a method of immobilizing a hydrophilic hydrogel on a metal surface via a linker so as to suppress physical adsorption has been applied (Japanese Patent No. 2815120, U.S. Pat. No. 5,436,161, and Japanese Patent Application Laid-Open No. 8-193948).

In order to eliminate influence caused by measurement disturbance (changes in temperature, in concentration, and in pressure) thereby reducing baseline fluctuation, it is preferable that a measurement unit for measuring a specific binding reaction between a physiologically active substance and a test substance and a reference unit wherein such a binding reaction is not carried out exist on a single plane of the above-described biosensor, and that such units be located as close as possible to each other. Thus, it became necessary to allow a reference unit and a measurement unit to coexist on an SPR sensor surface using a thin polymer film.

For example, International Publication W03/002985 describes that a ligand immobilization region (measurement unit) is separated from a ligand non-immobilization region (reference unit) via a laminar flow, and that an analyte is allowed to flow over them simultaneously. However, this method requires a complicated flow channel system and multiple steps. In addition, in the system described in International Publication W03/002985, a detection surface is not integrated with a prism, and all carboxylic acids existing in the ligand non-immobilization region cannot be blocked. Thus, this method has been problematic in that electric charge remains in the reference unit and in that the measurement performance is thereby deteriorated.

### DISCLOSURE OF INVENTION

It is an object of the present invention to solve the aforementioned problem. That is to say, it is an object of the present invention to provide a biosensor wherein a reference unit and a measurement unit can be prepared by performing only a single operation for immobilizing a physiologically active substance and wherein unnecessary electric charge does not remain in the reference unit.

As a result of intensive studies directed towards achieving the aforementioned object, the present inventors have found that a desired biosensor can be provided by establishing on a single plane of a substrate at least two types of surfaces, which include a surface for retaining a physiologically active substance and a surface that does not retain such a physiologically active substance, in a biosensor consisting of the substrate composed of a metal surface or metal film coated with a hydrophilic polymer compound, thereby completing the present invention.

Thus, the present invention provides a biosensor which comprises a substrate composed of a metal surface or metal film coated with a hydrophilic polymer compound, and which has a surface for retaining a physiologically active substance and a surface that does not retain a physiologically active substance on a single plane of the substrate.

Preferably, the biosensor according to the present invention has a surface having a functional group for binding a physiologically active substance and a surface that does not have a functional group for binding a physiologically active substance on a single plane of the substrate.

Preferably, the functional group for binding a physiologically active substance is a carboxyl group, an amino group, or a hydroxyl group.

Preferably, the biosensor according to the present invention has a surface having a carboxyl group as a surface for retaining a physiologically active substance, and which has a surface that does not have a carboxyl group and a blocked carboxyl group as a surface that does not retain a physiologically active substance.

Preferably, a hydrophilic polymer compound is immobilized on the substrate via a self-assembling film.

Preferably, the self-assembling film is formed from a sulfur-containing compound.

Preferably, the thickness of the swollen film of a hydrophilic polymer layer is between 10 m and 500 nm.

Preferably, the metal surface or metal film consists of a free electron metal selected from the group consisting of gold, silver, copper, platinum, and aluminum.

Preferably, the thickness of the metal film is between 0.5 nm and 500 nm.

Preferably, the biosensor according to the present invention is used in non-electrochemical detection, and is more preferably used in surface plasmon resonance analysis.

Preferably, the biosensor according to the present invention is formed in a measurement chip that is used for a surface plasmon resonance measurement device comprising a dielectric block, a metal film formed on one side of the dielectric block, a light source for generating a light beam, an optical system for allowing said light beam to enter said dielectric block so that total reflection conditions can be obtained at the interface between said dielectric block and said metal film and so that various incidence angles can be included, and a light-detecting means for detecting the state of surface plasmon resonance by measuring the intensity of the light beam totally reflected at said interface,

wherein said measurement chip is basically composed of said dielectric block and said metal film, wherein said dielectric block is formed as a block including all of an incidence face and an exit face for said light beam and a face on which said metal film is formed, and wherein said metal film is unified with this dielectric block.

Another aspect of the present invention provides a method for producing the biosensor according to the present invention, which comprises: a step of coating the substrate composed of a metal surface or metal film with a hydrophilic polymer compound; and a step of forming a surface for retaining a physiologically active substance and a surface that does not have a physiologically active substance on a single plane of the substrate, without allowing a solid to come into contact with a detection region.

Preferably, a diaphragm is used to form a surface for retaining a physiologically active substance and a surface that does not have a physiologically active substance on a single plane.

Further another aspect of the present invention provides a method for immobilizing a physiologically active substance on a biosensor, which comprises a step of allowing the biosensor according to the present invention to come into contact with a physiologically active substance, thereby binding said physiologically active substance to the surface of said biosensor via a covalent bond.

Preferably, a same treatment is performed on a surface for retaining a physiologically active substance and a surface that does not retain a physiologically active substance on the substrate, so as to allow the physiologically active substance to come into contact with the biosensor.

Further another aspect of the present invention provides a method for detecting or measuring a substance interacting with a physiologically active substance, which comprises a step of allowing a test substance to come into contact with the biosensor of the present invention to the surface of which the physiologically active substance binds via a covalent bond.

Preferably, a same treatment is performed on a surface for retaining a physiologically active substance and a surface that does not retain a physiologically active substance on the substrate, so as to allow a test substance to come into contact with the biosensor.

Preferably, the substance interacting with the physiologically active substance is detected or measured by a non-electrochemical method, and more preferably the substance interacting with the physiologically active substance is detected or measured by surface plasmon resonance analysis.

### BRIEF DESCRIPTION OF THE DRAWINGS

FIG. 1 shows a plastic prism used in examples.

FIG. 2 shows a diaphragm used in examples.

FIG. 3 shows a diaphragm used for patterning in examples.

FIG. 4 shows an SPR system used in examples.

### BEST MODE FOR CARRYING OUT THE INVENTION

The embodiments of the present invention will be described below.

The biosensor of the present invention is characterized in that it comprises a substrate composed of a metal surface or metal film coated with a hydrophilic polymer compound, and has a surface for retaining a physiologically active substance and a surface that does not retain a physiologically active substance on a single plane of the substrate.

In the present invention, the term “surface that does not retain a physiologically active substance” is used to mean a surface, the amount of a physiologically active substance retained on which is less than one tenth the amount of the above substance retained on a surface for retaining a physiologically active substance, when the surface has been treated to immobilize the physiologically active substance thereon (for example, the surface is treated with a mixture consisting of carboxylic acid activators, EDC and NHS, and then treated with a physiologically active substance).

A surface for retaining a physiologically active substance is preferably a surface, which has a functional group for binding the physiologically active substance. A surface that does not retain such a physiologically active substance is preferably a surface, which does not have a functional group for binding the physiologically active substance.

Specific examples of a functional group for binding a physiologically active substance may include —COOH, —NR¹R² (wherein each of R¹ and R² independently represents a hydrogen atom or a lower alkyl group), —OH, —SH, —CHO, —NR³NR¹R² (wherein each of R¹, R², and R³ independently represents a hydrogen atom or a lower alkyl group), —NCO, —NCS, an epoxy group, and a vinyl group. Herein, the number of carbon atoms contained in a lower alkyl group is not particularly limited. It is generally approximately C1 to C10, and preferably C1 to C6.

Preferred examples of such a functional group for binding a physiologically active substance may include a carboxyl group, an amino group, and a hydroxyl group.

A functional group for binding a physiologically active substance is selected depending on a method for immobilizing the physiologically active substance in the present invention. That is to say, a certain type of functional group (for example, a hydroxyl group, etc.) may be considered to be a “functional group for binding a physiologically active substance,” or may not considered to be such a functional group, depending on a method for immobilizing the physiologically active substance.

When a functional group for binding a physiologically active substance is a carboxyl group, for example, a method of generating an active ester by the combination use of carbodiimide with N-hydroxysuccinimide, and generating a covalent bond with an amino group of the physiologically active substance, is often used. In this case, a functional group incapable of binding a physiologically active substance, such as a hydroxyl group, an amino group, or polyethers, has been introduced into a surface which has no functional groups for binding such a physiologically active substance.

Moreover, when a functional group for binding a physiologically active substance is an amino group, a method of allowing glutaraldehyde to act thereon and then generating a covalent bond with an amino group of the physiologically active substance, and a method of oxidizing the physiologically active substance with periodate and then allowing the above substance to directly covalently bind to the amino group, are often used. In such cases, it may be possible that a functional group incapable of binding a physiologically active substance, such as a hydroxyl group, a carboxyl group, or polyethers, have been introduced into a surface which has no functional groups for binding such a physiologically active substance.

Furthermore, when a functional group for binding a physiologically active substance is a hydroxyl group, a method of allowing a polyepoxy compound or epichlorohydrin to act thereon and then generating a covalent bond with an amino group of the physiologically active substance is often used. As a chemical reaction, a direct ether bond formation reaction using halogenated alkyl is also applied. However, when such a reaction is applied to a physiologically active substance, there are cases where it becomes difficult to maintain the physiological activity. In such a case, it may be possible that a functional group incapable of binding a physiologically active substance, such as a water-soluble group (for example, a polyether such as polyethylene glycol), which has no hydrogen with reactivity (specifically, hydrogen of a hydroxyl group, an amino group, or a carboxyl group), have been introduced into a surface which has no functional groups for binding such a physiologically active substance.

When a surface for retaining a physiologically active substance and a surface that does not have a physiologically active substance are formed on a single plane of a substrate, it is preferable that a solid (for example, a stamp) be not allowed to come into contact with a detection region. Specific means may include a method of preparing a droplet on the tip of a syringe, so as to allow only such a droplet to come into contact with a detection region, a method of spraying droplets from a nozzle, a method of preparing a flow channel and flowing a reaction solution through it, and a method of establishing a diaphragm and filling it with a liquid. Of these, a method of using a diaphragm is preferable.

When the interaction between a physiologically active substance immobilized on the biosensor of the present invention and a test substance is measured, a surface for retaining a physiologically active substance in the biosensor is used as a measurement unit, whereas a surface that does not retain such a physiologically active substance is used as a reference unit. Further, by using several different substances as physiologically active substances to be bound, it may also be possible to establish multiple measurement units.

In the biosensor of the present invention, a substrate is coated with a hydrophilic polymer compound. An example of such a hydrophilic polymer used in the present invention is a biocompatible porous matrix such as a hydrogel. The thickness of such a biocompatible porous matrix is between several nm and several hundreds of nm, and preferably between 10 and 500 nm. An example of the hydrogel used in the present invention is a hydrogel described in Merrill et al. (1986), Hydrogels in Medicine and Pharmacy, vol. III, Chapter 1, CRC, edited by Peppas N A. Examples of such a hydrogel which can be used in the present invention may include: polysaccharides such as agarose, dextran, carragheenan, alginic acid, starch, cellulose; derivatives thereof such as a carboxymethyl derivative; and water-swellable organic polymers such as polyvinyl alcohol, polyacrylic acid, polyacrylamide, or polyethylene glycol. In particular, in contrast with cellulose, dextran-type polysaccharides have noncrystalline properties and are therefore preferably used.

The aforementioned hydrophilic polymer compound (preferably, a hydrogel) may be immobilized on a substrate via a self-assembling film as described below. Or, it may also be directly formed on a substrate from a solution containing a monomer. Further, it is also possible to crosslink the aforementioned hydrogel. Such crosslinking of a hydrogel is obvious to persons skilled in the art.

In the present invention, a self-assembling film is formed on a substrate, and thereafter, the surface thereof can be coated with a hydrophilic polymer compound. The term “self-assembling film” is used in the present invention to mean an ultra-thin film, such as a monomolecular film or an LB film, which has tissues with certain order formed by the mechanism of a film material itself in a state where no detailed controls are given from the outside. By such self-assembling, a structure or pattern with certain order can be formed over a long distance in a nonequilibrium situation.

For example, such a self-assembling film can be formed from a sulfur-containing compound. Formation of a self-assembling film from a sulfur-containing compound on a gold surface is described, for example, in Nuzzo R G et al. (1983), J. Am. Chem. Soc., vol. 105, pp. 4481-4483, Porter M D et al. (1987), J. Am. Chem. Soc., vol. 109, pp. 3559-3568, Troughton E B et al. (1988), Langmuir, vol. 4, pp. 365-385.

The sulfur-containing compound is preferably represented by X—R—Y.

X is a group having binding ability to a metal film. Specific examples of X, which is preferably used herein, may include asymmetric or symmetric sulfide (—SSR′Y″, —SSRY), sulfide (—SR′Y″, —SRY), diselenide (—SeSeR′Y″, —SeSeRY), selenide (SeR′Y″, —SeRY), thiol (—SH), nitrile (—CN), isonitrile, nitro (—NO₂), selenol (—SeH), a trivalent phosphorus compound, isothiocyanate, xanthate, thiocarbamate, phosphine, thio acid, and dithio acid (—COSH, —CSSH).

R (and R′) are blocked by heteroatoms in some cases. For suitably tight packing, R (and R′) are preferably linear (not branched) chains, and in some cases, are hydrocarbon chains containing double and/or triple bonds. The length of such a chain is generally 5 or more atoms, preferably 10 or more atoms, and more preferably 10 to 30 atoms. A carbon chain can be perfluoridated in some cases. When it is an asymmetric molecule, R′ or R may also be H.

Y and Y″ are groups for binding a hydrophilic polymer compound. Y and Y″ are preferably identical to each other, and they have properties such that they are able to bind to a hydrophilic polymer compound (for example, a hydrogel, etc.), directly or after activation. Specific examples of Y and Y″ that can be used herein may include a hydroxyl group, a carboxyl group, an amino group, an aldehyde group, a hydrazide group, group, a carbonyl group, an epoxy group, and a vinyl group.

The compound represented by X—R—Y, which is in the form of a tightly packed monolayer, is able to attach to the surface of a metal, by the binding of the group represented by X to the metal.

Specific examples of the compound represented by X—R—Y may include 10-carboxy-1-decanethiol, 4,4′-dithiodibutylic acid, 11-hydroxy-1-undecanethiol, 11-amino-1-undecanethiol, and 16-hydroxy-1-hexadecathiol.

The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

The biosensor of the present invention is obtained by coating a metal surface or metal film with a hydrophilic polymer compound. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 0.1 nm and 500 nm, more preferably between 0.5 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and have excellent workability are preferably used.

The biosensor of the present invention preferably has a functional group capable of immobilizing a physiologically active substance on the outermost surface of the substrate. The term “the outermost surface of the substrate” is used to mean “the surface, which is farthest from the substrate,” and more specifically, it means “the surface of a hydrophilic polymer compound applied on a substrate, which is farthest from the substrate.”

In order to introduce these functional groups into the outermost surface, a method is applied that involves applying a hydrophilic polymer compound containing a precursor of such a functional group on a metal surface or metal film, and then generating the functional group from the precursor located on the outermost surface by chemical treatment.

A physiologically active substance is covalently bound to the above-obtained surface for a biosensor via the above functional group, so that the physiologically active substance can be immobilized on the metal surface or metal film.

A physiologically active substance immobilized on the surface for the biosensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

When a physiologically active substance is a protein such as an antibody or enzyme or nucleic acid, an amino group, thiol group or the like of the physiologically active substance is covalently bound to a functional group located on a metal surface, so that the physiologically active substance can be immobilized on the metal surface.

A biosensor to which a physiologically active substance is immobilized as described above can be used to detect and/or measure a substance which interacts with the physiologically active substance.

Thus, the present invention provides a method of detecting and/or measuring a substance interacting with the physiologically active substance immobilized to the biosensor of the present invention, to which a physiologically active substance is immobilized, wherein the biosensor is contacted with a test substance.

As such a test substance, for example, a sample containing the above substance interacting with the physiologically active substance can be used.

In the present invention, it is preferable to detect and/or measure an interaction between a physiologically active substance immobilized on the surface used for a biosensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

The biosensor according to the present invention can be preferably formed and used in a measurement chip that is used for a surface plasmon resonance measurement device comprising a dielectric block, a metal film formed on one side of the dielectric block, a light source for generating a light beam, an optical system for allowing said light beam to enter said dielectric block so that total reflection conditions can be obtained at the interface between said dielectric block and said metal film and so that various incidence angles can be included, and a light-detecting means for detecting the state of surface plasmon resonance by measuring the intensity of the light beam totally reflected at said interface, wherein said measurement chip is basically composed of said dielectric block and said metal film, wherein said dielectric block is formed as a block including all of an incidence face and an exit face for said light beam and a face on which said metal film is formed, and wherein said metal film is unified with this dielectric block.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (θSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The present invention will be further specifically described in the following examples. However, the examples are not intended to limit the scope of the present invention.

### EXAMPLES

### Example 1

### Production of Chip of the Present Invention

The sensor chip of the present invention was produced by the following method.

(1) Formation of Gold Film on Plastic Prism

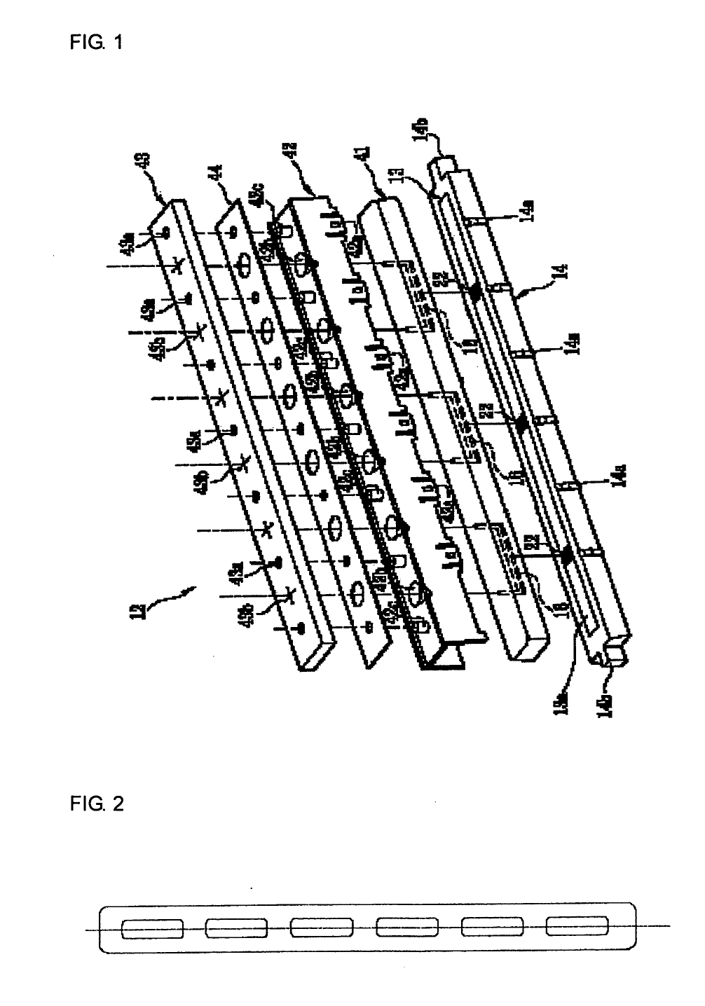

A thin gold film was formed on the top surface of a plastic prism (FIG. 1) obtained by the injection molding of ZEONEX (manufactured by JAPAN ZEON Corporation) by the following method.

(1-1) Formation of Gold Film

The prism was attached to the substrate holder of a sputter device. After vacuuming (base pressure: 1×10⁻³ Pa or less), Ar gas (1 Pa) was introduced therein. Thereafter, while rotating the substrate holder (20 rpm), RF power (0.5 kW) was applied to the substrate holder for approximately 9 minutes, so as to subject the surface of the prism to a plasma treatment (which is also referred to as substrate etching or reverse sputtering). After the application of such plasma, the surface roughness of the light reflection plane of an optical block was found to be Ra≦30 nm. Subsequently, introduction of Ar gas was terminated, followed by vacuuming. Thereafter, Ar gas was introduced again (0.5 Pa), and while rotating the substrate holder (10 to 40 rpm), DC power (0.2 kW) was applied to a Cr target with a size of 8 inch for approximately 30 seconds, so as to form a thin Cr film with a thickness of 2 nm. Subsequently, introduction of Ar gas was terminated, followed by vacuuming. Thereafter, Ar gas was introduced again (0.5 Pa), and while rotating the substrate holder (20 rpm), DC power (1 kW) was applied to an Au target with a size of 8 inch for approximately 50 seconds, so as to form a thin Au film with a thickness of approximately 50 nm. The particle size of Au was approximately 20 nm. The obtained sample was called chip A.

(1-2) Formation of Self-Assembling Film

Preparation of Solutions

SAM solution was produced by fully mixing 0.0102 g of 11-hydroxy-1-undecanethiol (manufactured by Dojindo Laboratories), 2 ml of ultrapure water, and 8 ml of ethanol. A washing solution was produced by fully mixing 2 ml of ultrapure water and 8 ml of ethanol.

Operations

A diaphragm having the shape shown in FIG. 2 was set in chip A, and 150 μl of the SAM solution was poured into each hole. While preventing evaporation, the sample was incubated with a shaking incubator at 40° C. for 30 minutes. Thereafter, the sample was removed and was then left at 25° C. for 16 hours. After leaving, the sample was washed with the washing solution 15 times for displacement washing. The obtained sample was called chip B. Taking care of not drying the surface, the chip with the above diaphragm was subjected to the next operation.

(1-3) Production of Hydrogel Layer

Preparation of Solutions

An epichlorohydrin solution was produced by fully mixing 500 μl of epichlorohydrin (manufactured by Wako Pure Chemical Industries, Ltd.), 4.5 ml of diethylene glycol dimethyl ether, 3 ml of ultrapure water, and 2 ml of 1 mol/L NaOH. A dextran solution was produced by fully mixing 3 g of dextran T-500 (manufactured by Amersham), 9 ml of ultrapure water, and 1 ml of 1 mol/L NaOH.

Operations

150 μl of the epichlorohydrin solution was poured into each hole of chip B in which a diaphragm had been equipped. While preventing evaporation, the sample was incubated with a shaking incubator at 25° C. for 4 hours. Thereafter, the sample was removed and was then left. Thereafter, the sample was washed with ethanol 10 times and then with ultrapure water 5 times for displacement washing. Thereafter, the washing solution was removed, and 150 μl of the dextran solution was then poured into each hole. While preventing evaporation, the sample was incubated with a shaking incubator at 25° C. for 20 hours. Thereafter, the sample was removed and was then washed with ultrapure water at 60° C. 15 times for displacement washing. The obtained sample was called chip C.

(1-4) Patterning (Carboxymethylation)

Preparation of Solution

A bromoacetic acid solution was produced by fully mixing 1.2 g of bromoacetic acid, 5.4 ml of ultrapure water, and 3.2 ml of 5 mol/L NaOH.

Operations

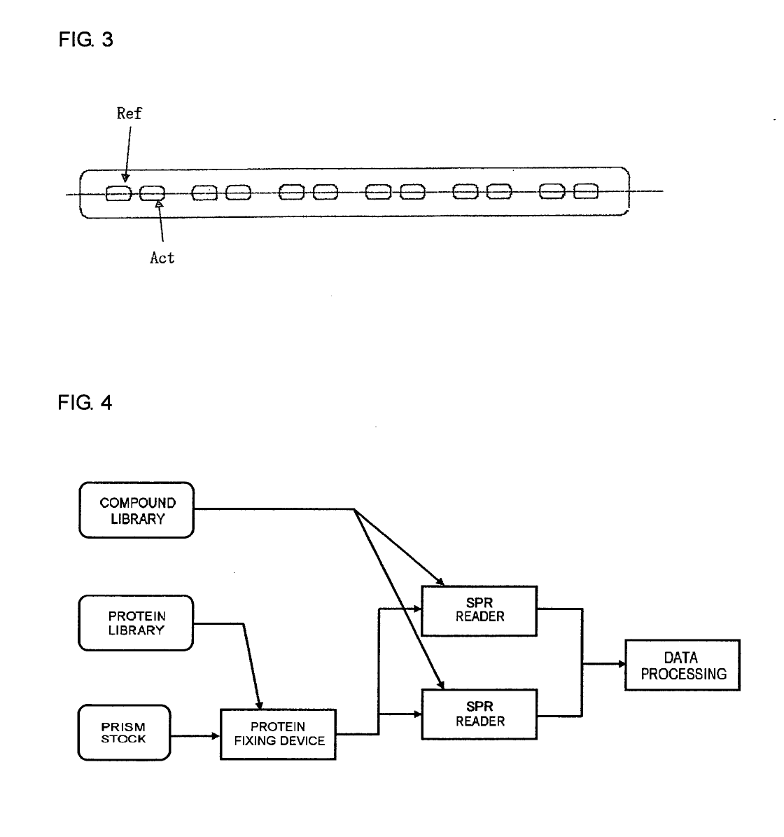

A diaphragm having the shape shown in FIG. 3 was set in chip C. Thereafter, 100 μl each of the bromoacetic acid solution was poured into only holes, which were located on the side for immobilizing a physiologically active substance. On the other hand, 100 μl each of ultrapure water was poured into holes, which were located on the side that did not immobilize a physiologically active substance. While preventing evaporation, the sample was incubated with a shaking incubator at 25° C. for 16 hours. Thereafter, the sample was washed with ultrapure water 5 times, and the same operations as those described above were carried out thereon. Thereafter, the resultant was washed with ultrapure water 10 times. The obtained sample was called chip D.

### Example 2

Chip B was produced by the same method as that described in Example 1. Thereafter, the following patterning was carried out.

Preparation of Solutions

An epichlorohydrin solution was produced by fully mixing 500 μl of epichlorohydrin (manufactured by Wako Pure Chemical Industries, Ltd.), 4.5 ml of diethylene glycol dimethyl ether, 3 ml of ultrapure water, and 2 ml of 1 mol/L NaOH. A dextran solution was produced by fully mixing 3 g of dextran T-500 (manufactured by Amersham), 9 ml of ultrapure water, and 1 ml of 1 mol/L NaOH. A carboxymethyl dextran solution was produced by mixing 3 g of carboxymethyl dextran (1 carboxylic acid introduction rate per unit), 8 ml of ultrapure water, and 2 ml of 1 mol/L NaOH.

Operations

The diaphragm shown in FIG. 3 was set in chip B. Thereafter, 150 μl of the epichlorohydrin solution was poured into each hole thereof. While preventing evaporation, the sample was incubated with a shaking incubator at 25° C. for 4 hours. Thereafter, the sample was removed and was then left. Thereafter, the sample was washed with ethanol 10 times and then with ultrapure water 5 times for displacement washing. Thereafter, the washing solution was removed, and 150 μl each of the carboxymethyl dextran solution was poured into holes in a region for immobilizing a physiologically active substance. On the other hand, 150 μl each of the dextran solution was poured into holes in a region which did not immobilize such a physiologically active substance. While preventing evaporation, the sample was incubated with a shaking incubator at 25° C. for 20 hours. Thereafter, the sample was removed and was then washed with ultrapure water at 60° C. 15 times for displacement washing. The obtained sample was called chip E.

### Comparative Example 1

### Production of Chip of Comparative Example

The sensor chip of the comparative example was produced by the following method.

(1) Formation of Gold Film on Plastic Prism

A thin gold film was formed on the top surface of a plastic prism (FIG. 1) obtained by the injection molding of ZEONEX (manufactured by JAPAN ZEON Corporation) by the following method.

(1-1) Formation of Gold Film

The prism was attached to the substrate holder of a sputter device. After vacuuming (base pressure: 1×10⁻³ Pa or less), Ar gas (1 Pa) was introduced therein. Thereafter, while rotating the substrate holder (20 rpm), RF power (0.5 kW) was applied to the substrate holder for approximately 9 minutes, so as to subject the surface of the prism to a plasma treatment (which is also referred to as substrate etching or reverse sputtering). After the application of such plasma, the surface roughness of the light reflection plane of an optical block was found to be Ra≦30 nm. Subsequently, introduction of Ar gas was terminated, followed by vacuuming. Thereafter, Ar gas was introduced again (0.5 Pa), and while rotating the substrate holder (10 to 40 rpm), DC power (0.2 kW) was applied to a Cr target with a size of 8 inch for approximately 30 seconds, so as to form a thin Cr film with a thickness of 2 nm. Subsequently, introduction of Ar gas was terminated, followed by vacuuming. Thereafter, Ar gas was introduced again (0.5 Pa), and while rotating the substrate holder (20 rpm), DC power (1 kW) was applied to an Au target with a size of 8 inch for approximately 50 seconds, so as to form a thin Au film with a thickness of approximately 50 nm. The particle size of Au was approximately 20 nm. The obtained sample was called chip A2.

(1-2) Formation of Self-Assembling Film

Preparation of Solutions

SAM solution was produced by fully mixing 0.0102 g of 11-hydroxy-1-undecanethiol (manufactured by Dojindo Laboratories), 2 ml of ultrapure water, and 8 ml of ethanol. A washing solution was produced by fully mixing 2 ml of ultrapure water and 8 ml of ethanol.

Operations

A diaphragm having the shape shown in FIG. 2 was set in chip A, and 150 μl of the SAM solution was poured into each hole. While preventing evaporation, the sample was incubated with a shaking incubator at 40° C. for 30 minutes. Thereafter, the sample was removed and was then left at 25° C. for 16 hours. After leaving, the sample was washed with the washing solution 15 times for displacement washing. The obtained sample was called chip B2. Taking care of not drying the surface, the chip with the above diaphragm was subjected to the next operation.

(1-3) Production of Hydrogel Layer

Preparation of Solutions

An epichlorohydrin solution was produced by fully mixing 500 μl of epichlorohydrin (manufactured by Wako Pure Chemical Industries, Ltd.), 4.5 ml of diethylene glycol dimethyl ether, 3 ml of ultrapure water, and 2 ml of 1 mol/L NaOH. A dextran solution was produced by fully mixing 3 g of dextran T-500 (manufactured by Amersham), 9 ml of ultrapure water, and 1 ml of 1 mol/L NaOH.

Operations

150 μl of the epichlorohydrin solution was poured into each hole of chip B in which a diaphragm had been equipped. While preventing evaporation, the sample was incubated with a shaking incubator at 25° C. for 4 hours. Thereafter, the sample was removed and was then left. Thereafter, the sample was washed with ethanol 10 times and then with ultrapure water 5 times for displacement washing. Thereafter, the washing solution was removed, and 150 μl of the dextran solution was then poured into each hole. While preventing evaporation, the sample was incubated with a shaking incubator at 25° C. for 20 hours. Thereafter, the sample was removed and was then washed with ultrapure water at 60° C. 15 times for displacement washing. The obtained sample was called chip C2. Taking care of not drying the surface, the chip with the above diaphragm was subjected to the next operation.

(1-4) Carboxymethylation

Preparation of Solution

A bromoacetic acid solution was produced by fully mixing 1.2 g of bromoacetic acid, 5.4 ml of ultrapure water, and 3.2 ml of 5 mol/L NaOH.

Operations

100 μl each of the bromoacetic acid solution was poured into each hole of chip C2 equipped with a diaphragm. On the other hand, 150 μl each of ultrapure water was poured into holes which were located on the side that did not immobilize a physiologically active substance. While preventing evaporation, the sample was incubated with a shaking incubator at 25° C. for 16 hours. Thereafter, the sample was washed with ultrapure water 5 times, and the same operations as those described above were carried out thereon again. Thereafter, the resultant was washed with ultrapure water 10 times. The obtained sample was called chip D2.

(1-5) Patterning

Preparation of Solutions

Activating solution (0.1 M NHS solution was mixed with 0.4 M EDC solution at a ratio of 1:1 (volume ratio) immediately before use.)

0.1 M NHS solution: 1.16 g of NHS(N-hydroxysuccinimide) was dissolved in ultrapure water to a final volume of 100 ml.

0.4 M EDC solution: 7.7 g of EDC (1-ethyl-3-(3-dimethylaminopropyl)carbodiimide hydrochloride) was dissolved in ultrapure water to a final volume of 100 ml.

Immediately before use, 0.1 M NHS solution was mixed with 0.4 M EDC solution at a ratio of 1:1 (volume ratio).

1 M ethanolamine solution: 9.76 g of ethanolamine-1-hydrochloride was dissolved in 90 ml of ultrapure water, and the pH thereof was then adjusted to pH 8.5 by addition of 1 N NaOH. Thereafter, ultrapure water was added thereto to a final volume of 100 ml.

Operations

A diaphragm having the shape shown in FIG. 3 was set in chip D2. Thereafter, 100 μl each of the activating solution was poured into only holes which were located on the side that did not immobilize a physiologically active substance. On the other hand, 100 μl each of ultrapure water was poured into holes which were located on the side for immobilizing a physiologically active substance. While preventing evaporation, the sample was incubated with a shaking incubator at 25° C. for 30 minutes. Thereafter, the sample was washed with ultrapure water twice, and 100 μl each of the ethanolamine solution was poured into only holes which were located on the side that did not immobilize a physiologically active substance. On the other hand, 100 μl each of ultrapure water was poured into holes which were located on the side for immobilizing a physiologically active substance. The sample was washed with ultrapure water 5 times. The obtained chip was called chip F.

### Test Example 1

### Measurement of Amount of Ligand Immobilized

A ligand protein was immobilized on each of chip D (the present invention), chip E (the present invention), and chip F (comparative example), to which the aforementioned treatments had been performed, according to the following method. Thereafter, an analyte was flown thereon, so as to evaluate the binding amount. The amount of the protein immobilized was measured using the SPR device shown in FIG. 4. For the measurement, the flow channel made from Taffcellen of part 41 shown in FIG. 1 was used.

(1) Preparation of ligand solution: 0.5 mg of CA (carbonic anhydrase) (manufactured by Sigma) was dissolved in 1 ml of an acetate buffer (pH 5.5).

(2) Preparation of activating solution: The following solutions were mixed at a volume ratio of 1:1 immediately before use.

0.1 M NHS solution, 0.4 M EDC solution (For preparation of these solutions, refer to Comparative example 1.)

(3) Blocking solution: 1 M ethanolamine solution (pH 8.5) (For preparation of this solution, refer to Comparative example 1.)

(4) Buffer for analyte measurement:

20 ml of ×10 PBS (pH 7.4) (manufactured by Wako Pure Chemical Industries, Ltd.), 10 ml of DMSO, and 170 ml of ultrapure water were fully mixed, so as to prepare a buffer used for analyte measurement.

(5) Analyte solution

2.95 mg of chlorothiazide (manufactured by Sigma) was dissolved in 10 ml of DMSO, so as to prepare solution (A). Thereafter, 9.99 ml of the buffer for analyte measurement was added to 10 μl of solution (A).

A chip was set in a device, and the flow channel thereof was filled with an HBS-EP buffer. It is to be noted that the HBS-EP buffer consisted of 0.01 mol/L HEPES (N-2-hydroxyethylpiperazine-N′-2-ethanesulfonic acid) (pH 7.4), 0.15 mol/L NaCl, 0.003 mol/L EDTA, and 0.005% by weight of Surfactant P20. The measurement of a ligand immobilization unit (Act) and that of a ligand non-immobilization unit (Ref) were carried out simultaneously. The measurement was initiated in such a state, and the signal value obtained 30 seconds after initiation of the measurement was defined as 0. While the measurement was continued, 100 μl of the activating solution was poured into the flow channel for 1 second, and it was then left for 15 minutes. Subsequently, 100 μl of the HBS-EP buffer was poured into the flow channel for 1 second, and 100 μl of the ligand solution was then poured into the flow channel for 1 second. It was left for 15 minutes. Thereafter, 100 μl of the HBS-EP buffer was poured into the flow channel for 1 second, and 100 μl of the blocking solution was then poured into the flow channel for 1 second. It was left for 15 minutes. Thereafter, the operation to pour 100 μl of the HBS-EP buffer into the flow channel for 1 second and then to pour 100 μl of the 10 mM NaOH solution therein for 1 second was repeated twice, followed by substitution with HBS-EP. The resultant was then left for 30 seconds. The obtained signal value was defined as an amount immobilized.

The above chip was still set in the above device, and the analyte was measured. The measurement of a ligand immobilization unit (Act) and that of a ligand non-immobilization unit (Ref) were carried out simultaneously. The flow channel was filled with the buffer for analyte measurement, and the measurement was initiated in such a state. The signal value obtained 60 seconds after initiation of the measurement was defined as 0. While the measurement was continued, 100 μl of the analyte solution was poured into the flow channel for 1 second, and it was then left for 3 minutes. The signal value obtained 3 minutes later was measured. In order to eliminate the bulk effect, the value of Act-Ref was defined as an amount bound. The results are shown in Table 1.

TABLE 1

| | Ligand | | | |
|---|---|---|---|---|
| | immobilization | | Analyte binding | |
| | amount | | amount | |
| | Act | Ref | Act-Ref | Remarks |
| Chip D | 7150 | 10< | 60 | The present invention |
| Chip E | 6800 | 10< | 55 | The present invention |
| Chip F | 7200 | 1050 | 35 | Comparative example |

The existence of carboxylic acid in the Ref unit causes a problem in that a ligand is immobilized even if a blocking treatment has been carried out. Thus, the amount of the analyte bound also decreases. Ref with good performance can be produced by the method of the present invention.

### EFFECTS OF THE INVENTION

By using the biosensor of the present invention, a reference unit and a measurement unit can be prepared by performing only a single operation of immobilizing a physiologically active substance. In addition, since unnecessary electric charge cannot remain in a reference unit in the biosensor of the present invention, adsorption of a physiologically active substance (ligand) on the reference unit can be reduced to a minimum. Thus, a biosensor having excellent performance can be provided.

1. A biosensor which comprises a substrate composed of a metal surface or metal film coated with a hydrophilic polymer compound, and which has a surface for retaining a physiologically active substance and a surface that does not retain a physiologically active substance on a single plane of the substrate.

2. The biosensor according to claim 1, which has a surface having a functional group for binding a physiologically active substance and a surface that does not have a functional group for binding a physiologically active substance on a single plane of the substrate.

3. The biosensor according to claim 2, wherein the functional group for binding a physiologically active substance is a carboxyl group, an amino group, or a hydroxyl group.

4. The biosensor according to claim 1, which has a surface having a carboxyl group as a surface for retaining a physiologically active substance, and which has a surface that does not have a carboxyl group and a blocked carboxyl group as a surface that does not retain a physiologically active substance.

5. The biosensor according to claim 1, wherein a hydrophilic polymer compound is immobilized on the substrate via a self-assembling film.

6. The biosensor according to claim 5, wherein the self-assembling film is formed from a sulfur-containing compound.

7. The biosensor according to claim 1, wherein the thickness of the swollen film of a hydrophilic polymer layer is between 10 nm and 500 nm.

8. The biosensor according to claim 1, wherein the metal surface or metal film consists of a free electron metal selected from the group consisting of gold, silver, copper, platinum, and aluminum.

9. The biosensor of claim 1, wherein the thickness of the metal film is between 0.5 nm and 500 nm.

10. The biosensor according to claim 1, which is used in non-electrochemical detection.

11. The biosensor according to claim 1, which is used in surface plasmon resonance analysis.

12. The biosensor according to claim 1, which is formed in a measurement chip that is used for a surface plasmon resonance measurement device comprising a dielectric block, a metal film formed on one side of the dielectric block, a light source for generating a light beam, an optical system for allowing said light beam to enter said dielectric block so that total reflection conditions can be obtained at the interface between said dielectric block and said metal film and so that various incidence angles can be included, and a light-detecting means for detecting the state of surface plasmon resonance by measuring the intensity of the light beam totally reflected at said interface,
wherein said measurement chip is basically composed of said dielectric block and said metal film, wherein said dielectric block is formed as a block including all of an incidence face and an exit face for said light beam and a face on which said metal film is formed, and wherein said metal film is unified with this dielectric block.

13. A method for producing the biosensor according to claim 1, which comprises: a step of coating the substrate composed of a metal surface or metal film with a hydrophilic polymer compound; and a step of forming a surface for retaining a physiologically active substance and a surface that does not have a physiologically active substance on a single plane of the substrate, without allowing a solid to come into contact with a detection region.

14. The method for producing the biosensor according to claim 13, wherein a diaphragm is used to form a surface for retaining a physiologically active substance and a surface that does not have a physiologically active substance on a single plane.

15. A method for immobilizing a physiologically active substance on a biosensor, which comprises a step of allowing the biosensor according to claim 1 to come into contact with a physiologically active substance, thereby binding said physiologically active substance to the surface of said biosensor via a covalent bond.

16. The method according to claim 15, wherein a same treatment is performed on a surface for retaining a physiologically active substance and a surface that does not retain a physiologically active substance on the substrate, so as to allow the physiologically active substance to come into contact with the biosensor.

17. A method for detecting or measuring a substance interacting with a physiologically active substance, which comprises a step of allowing a test substance to come into contact with the biosensor of claim 1 to the surface of which the physiologically active substance binds via a covalent bond.

18. The method according to claim 17, wherein a same treatment is performed on a surface for retaining a physiologically active substance and a surface that does not retain a physiologically active substance on the substrate, so as to allow a test substance to come into contact with the biosensor.

19. The method of claim 17, wherein the substance interacting with the physiologically active substance is detected or measured by a non-electrochemical method.

20. The method of claim 17, wherein the substance interacting with the physiologically active substance is detected or measured by surface plasmon resonance analysis.
